(19) United States
(12) Patent Application Publication
(10) Pub. No.: US 2022/0292389 A1
(43) Pub. Date: Sep. 15, 2022

(54) BIOINFORMATICS PROCESSING ORCHESTRATION

(71) Applicant: International Business Machines Corporation, Armonk, NY (US)

(72) Inventors: Edward E. Seabolt, Georgetown, TX (US); Kristen Beck, San Jose, CA (US); Vadim Elisseev, Warrington (GB); Ritesh Vijay Krishna, Sale (GB)

(21) Appl. No.: 17/197,098

(22) Filed: Mar. 10, 2021

(51) Int. Cl. G06N 20/00

(52) U.S. Cl. CPC G06N 20/00

(57) ABSTRACT

Computer software that performs the following operations: (i) identifying a bioinformatics dataset and instructions for processing the bioinformatics dataset, the instructions identifying a sequence of bioinformatics processing tools including at least a first bioinformatics processing tool followed by a second bioinformatics processing tool; (ii) instructing the first bioinformatics processing tool to process the bioinformatics dataset in accordance with the instructions; (iii) analyzing an output of the first bioinformatics processing tool, utilizing a machine learning based decision model, to determine a modification to the sequence of bioinformatics processing tools; and (iv) instructing a third bioinformatics processing tool to process at least a first portion of the bioinformatics dataset in accordance with the determined modification.

### BACKGROUND

The present invention relates generally to the field of bioinformatics, and more particularly to bioinformatics processing pipeline orchestration.

The rise of genomics in life sciences due to advancements in instrumentation technologies has resulted in ever growing amounts of data to processed. One way of processing such data to derive actionable insights is through bioinformatics pipelines. Bioinformatics pipelines generally include multiple tools that exchange data with each other and are connected in a relatively linear/deterministic workflow.

### SUMMARY

According to an aspect of the present invention, there is a method, computer program product and/or system that performs the following operations (not necessarily in the following order): (i) identifying a bioinformatics dataset and instructions for processing the bioinformatics dataset, the instructions identifying a sequence of bioinformatics processing tools including at least a first bioinformatics processing tool followed by a second bioinformatics processing tool; (ii) instructing the first bioinformatics processing tool to process the bioinformatics dataset in accordance with the instructions; (iii) analyzing an output of the first bioinformatics processing tool, utilizing a machine learning based decision model, to determine a modification to the sequence of bioinformatics processing tools; and (iv) instructing a third bioinformatics processing tool to process at least a first portion of the bioinformatics dataset in accordance with the determined modification.

### BRIEF DESCRIPTION OF THE DRAWINGS

FIG. 1 depicts a cloud computing node used in a first embodiment of a system according to the present invention;

FIG. 2 depicts an embodiment of a cloud computing environment (also called the “first embodiment system”) according to the present invention;

FIG. 3 depicts abstraction model layers used in the first embodiment system;

FIG. 4 is a flowchart showing a first embodiment method performed, at least in part, by the first embodiment system;

FIG. 5 is a block diagram showing a machine logic (for example, software) portion of the first embodiment system;

FIG. 6A is a block diagram showing an initial bioinformatics pipeline, according to the first embodiment system;

FIG. 6B is a block diagram showing a modified bioinformatics pipeline, according to the first embodiment system;

FIG. 7 is a block diagram showing a bioinformatics pipeline, according to an embodiment of the present invention;

FIG. 8 is a block diagram showing a bioinformatics pipeline with an orchestration engine, according to an embodiment of the present invention;

FIG. 9 is a block diagram showing another bioinformatics pipeline with an orchestration engine, according to an embodiment of the present invention; and

FIG. 10 is block diagram showing an orchestration engine architecture, according to an embodiment of the present invention.

### DETAILED DESCRIPTION

Existing bioinformatics pipelines and/or workflows do not allow modifications to be made during run time, resulting in long execution times and wasted computational resources. Embodiments of the present invention improve upon existing bioinformatics pipelines by providing intelligent bioinformatics pipelines, enabled via machine learning, that have dynamic, self-morphing workflows. This Detailed Description section is divided into the following sub-sections: (i) The Hardware and Software Environment; (ii) Example Embodiment; (iii) Further Comments and/or Embodiments; and (iv) Definitions.

### I. The Hardware and Software Environment

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based email). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

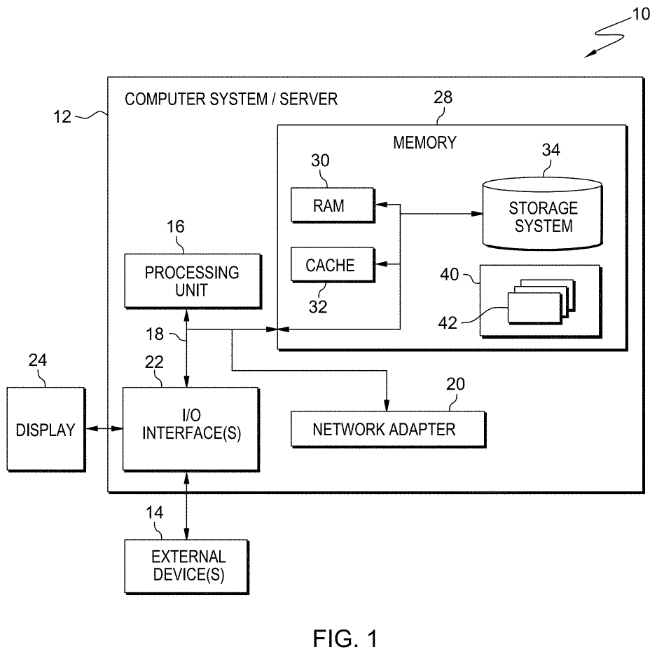

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples include, but are not limited to: microcode, device drivers, redundant processing units, external disk drive arrays, RAID systems, tape drives, and data archival storage systems, etc.

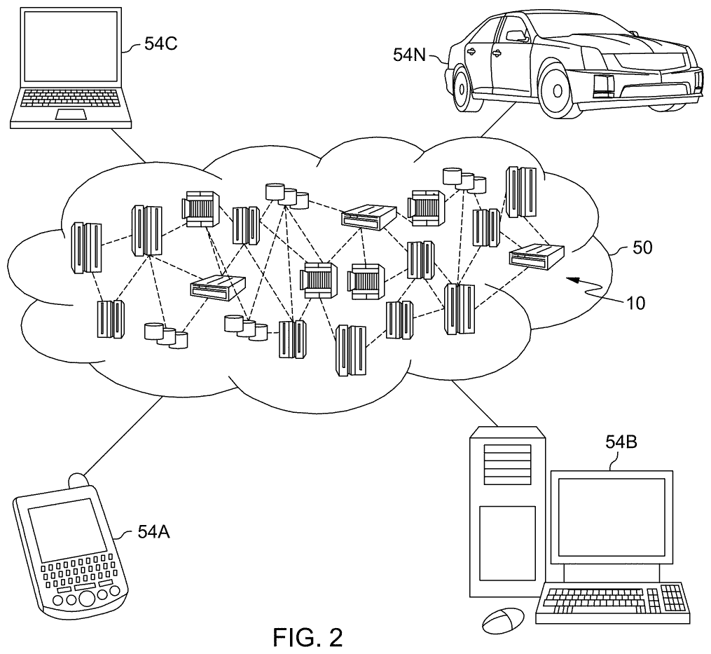

Referring now to FIG. 2, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

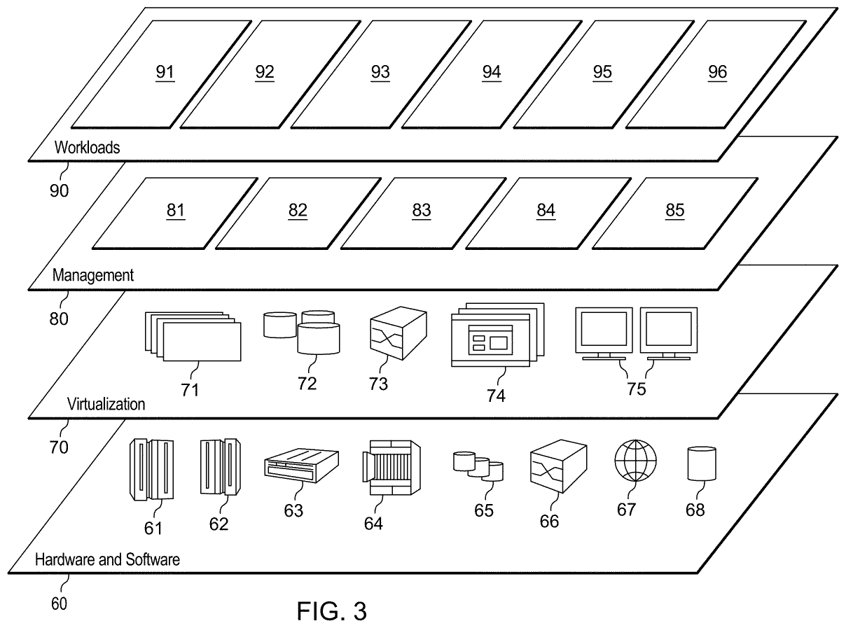

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and functionality according to the present invention 96, as will be discussed in detail, below, in the following sub-sections of this Detailed description section.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### II. Example Embodiment

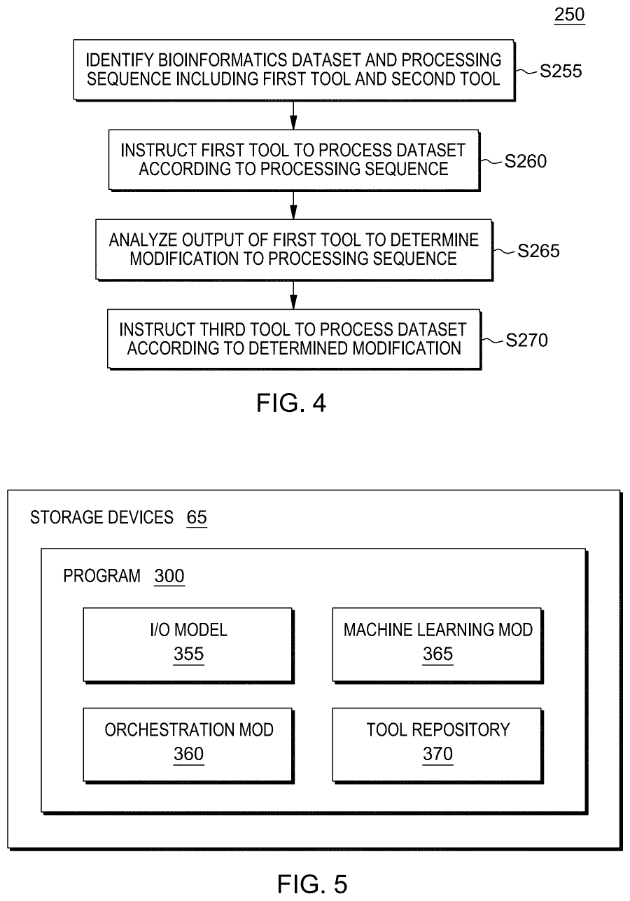

FIG. 4 shows flowchart 250 depicting a method according to the present invention. FIG. 5 shows program 300 for performing at least some of the method operations of flowchart 250. This method and associated software will now be discussed, over the course of the following paragraphs, with extensive reference to FIG. 4 (for the method operation blocks) and FIG. 5 (for the software blocks). One physical location where program 300 of FIG. 5 may be stored is in storage devices 65 (see FIG. 3).

Generally speaking, in this example embodiment (also referred to in this sub-section as the “present embodiment,” the “present example,” the “present example embodiment,” and the like), program 300 performs various operations relating to a bioinformatics dataset. It should be noted that this example embodiment is used herein for example purposes, in order to help depict the scope of the present invention. As such, other embodiments (such as embodiments discussed in the Further Comments and/or Embodiments sub-section, below) may be configured in different ways or refer to other features, advantages, and/or characteristics not fully discussed in this sub-section.

As used herein, a bioinformatics dataset is any computer readable dataset that relates to or represents biological data, including, for example, data relating to molecular biology, genomics, biochemistry, cellular biology, metabolomics, and related fields. Some example file formats for bioinformatics datasets include, but certainly aren't limited to, FASTA, FASTQ, SAM, and/or GVF files.

Bioinformatics pipelines, as used herein, are combinations—sometimes in a particular sequence—of bioinformatics processing tools that are used to analyze and interpret bioinformatics data in particular ways. For example, a bioinformatics pipeline that will be discussed in this sub-section involves genome annotation: it receives a genomic dataset (that is, nucleotide sequence data representing an assembled genome with genetic, intergenic, and other genomic features) and processes the genomic dataset using several gene annotation tools arranged in a certain order (or “sequence”). However, the operations described in this sub-section and in the context of the present embodiment are not specifically limited to genome annotation, and can be applied to many of a wide variety of bioinformatic activities, including, but certainly not limited to sequence alignment, drug design, protein structure prediction, evolution modeling, and the like.

Processing begins at operation 5255, where I/O module (“mod”) 355 identifies a bioinformatics dataset and instructions for processing the bioinformatics dataset. In this embodiment, the instructions for processing the bioinformatics dataset include a configuration file that provides a basic outline of a bioinformatics pipeline, including a short, predefined sequence of bioinformatics processing tools: a first bioinformatics processing tool followed by a second bioinformatics processing tool.

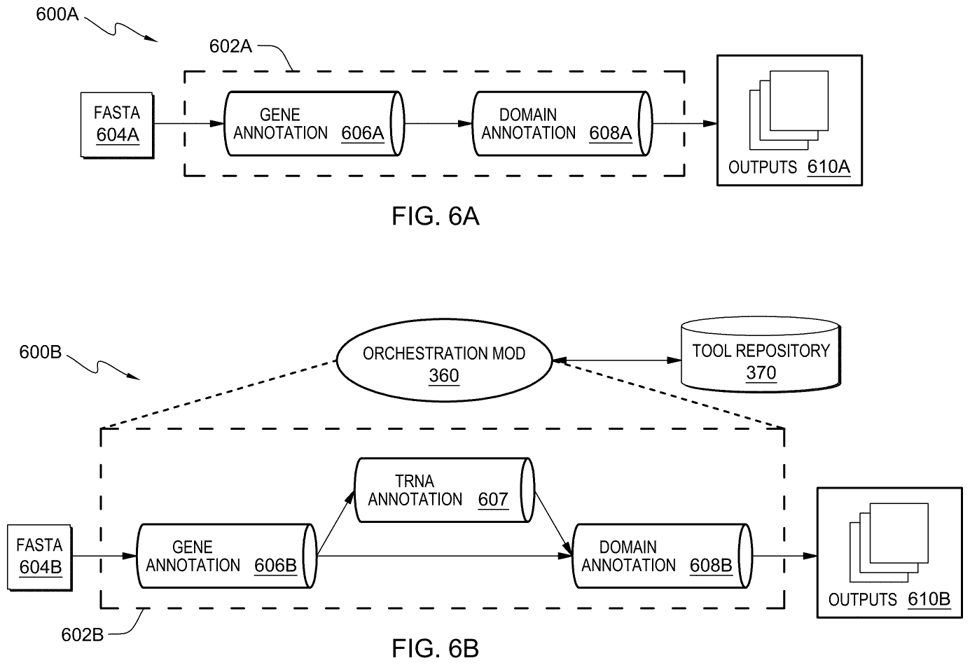

An example of an initial bioinformatics pipeline in accordance with this embodiment is depicted in FIG. 6A. As shown in FIG. 6A, bioinformatics pipeline 600A includes FASTA 604A, processing sequence 602A, which includes gene and protein annotation tool 606A and domain annotation tool 608A, and outputs 610A. In this example, FASTA 604A is the bioinformatics dataset, a genomic dataset encoded in FASTA format, and tools 606A and 608A are the bioinformatics tools outlined in the configuration file, where tool 606A is the Prokka tool and tool 608A is the InterProScan tool.

An example configuration file follows:

Example Configuration File
// Configuration File
name: Genome Annotation
tools:
-:
name: Prokka
command: <command>
-:
name: InterProScan
command: <command>

As shown, the Example Configuration File identifies the bioinformatics processing tools to be used in processing the genetic sequence of the bioinformatics dataset. It should also be noted that the list of tools in the Example Configuration File is ordered: the file dictates that the first bioinformatics tool (Prokka) process the bioinformatics dataset first, followed by the second bioinformatics tool (InterProScan) in sequence.

Processing proceeds to operation S260, where orchestration mod 360 instructs the first bioinformatics processing tool to process the bioinformatics dataset in accordance with the identified instructions. In this operation, mod 360 simply references the processing sequence in the configuration file and identifies the first bioinformatics processing tool (e.g., gene and protein annotation tool 606A) as the first tool to use in processing the bioinformatics dataset. However, in other embodiments, other, more complex operations may take place here. For example, in some embodiments, orchestration mod 360 uses a trained machine learning model and bioinformatics processing tool repository—such as it does in operation, S265, discussed below—to determine how to begin processing of the bioinformatics dataset.

Processing proceeds to operation S265, where machine learning mod 365 analyzes an output of the first bioinformatics processing tool, utilizing a machine learning based decision model, to determine a modification to the processing sequence of bioinformatics processing tools. Many types of modifications to the processing sequence may be made, many of which include the incorporation of one or more additional bioinformatics processing tools. For example, in various embodiments, the modification replaces the second bioinformatics processing tool in the sequence of bioinformatics processing tools with a third bioinformatics processing tool for at least a first portion of the bioinformatics dataset. In other embodiments, the modification adds the third bioinformatics processing tool to the sequence of bioinformatics processing tools after the first bioinformatics processing tool and before the second bioinformatics processing tool for at least the first portion of the bioinformatics dataset. In still other embodiments, the modification adds a branch to the sequence of bioinformatics processing tools, where the branch instructs parallel processing of at least the first portion of the bioinformatics dataset by both the second bioinformatics processing tool and the third bioinformatics processing tool. Still yet, other, more complex modifications may be made, with the general rule being that the modifications modify the sequence of bioinformatics processing tools in at least some way.

An example of a modified bioinformatics pipeline in accordance with this embodiment is depicted in FIG. 6B. As shown in FIG. 6B, modified bioinformatics pipeline 600B includes FASTA 604B, processing sequence 602B, which now includes gene and protein annotation tool 606B and domain annotation tool 608B, but also tRNA annotation tool 607, and outputs 610B. FIG. 6B also shows orchestration mod 360 and bioinformatics tool repository 370 for reference. As shown, tRNA annotation tool 607 has been inserted between gene and protein annotation tool 606B and domain annotation tool 608B in processing sequence 602B, causing at least some of the data from the bioinformatics dataset to be first processed by tRNA annotation tool 607 prior to processing by domain annotation tool 608B.

Orchestration mod 360 may utilize a wide variety of information and/or techniques in determining to modify the sequence of bioinformatics processing tools. As mentioned above, this determination is made, at least in part, utilizing a machine learning based decision model. In this embodiment, the decision model is an artificial intelligence (AI) classifier that has been trained by machine learning mod 365 utilizing historical bioinformatics datasets and bioinformatics processing tool repository 370. While many additional details about the training of the AI classifier are discussed below in the Further Comments and/or Embodiments sub-section of this Detailed Description, it should at least be noted here that in many cases, the AI classifier is trained to identify additional bioinformatics processing tools, beyond the bioinformatics processing tools included in the sequence of bioinformatics processing tools, that may be helpful for processing a particular bioinformatics dataset. By identifying additional bioinformatics processing tools beyond those initially anticipated for a given bioinformatics pipeline, orchestration mod 360 is able to dynamically modify the bioinformatics pipeline in real time, effectively morphing the pipeline to meet various needs and/or objectives not previously incorporated into the configuration file.

Processing proceeds to operation S270, where orchestration mod 360 instructs a third bioinformatics processing tool to process at least a first portion of the bioinformatics dataset in accordance with the determined modification. As mentioned above, in some cases, the modification replaces the second bioinformatics processing tool with the third bioinformatics processing tool for at least a portion of the bioinformatics dataset. In these cases, processing may complete after the third bioinformatics tool completes execution, or orchestration mod 360 may analyze the output of the third bioinformatics tool to determine whether additional bioinformatics processing tools should be utilized. Additionally, in the case where not all of the bioinformatics dataset is sent to the third bioinformatics tool for processing, a remaining portion of the bioinformatics dataset may be send to the second bioinformatics processing tool or elsewhere. In still other cases, where the modification adds the third bioinformatics processing tool to the sequence of bioinformatics processing tools after the first bioinformatics processing tool and before the second bioinformatics processing tool, when the third bioinformatics processing tool completes execution, orchestration mod 360 instructs the second bioinformatics processing tool to process the bioinformatics dataset, potentially utilizing the output of the third bioinformatics processing tool. In yet other cases, where the modification adds a branch to the sequence of bioinformatics tools, orchestration mod 360 instructs the third bioinformatics processing tool to process the bioinformatics dataset in parallel with the second bioinformatics processing tool.

Referring again to FIG. 6B, in this example, orchestration mod 360 has modified processing sequence 602B by inserting tRNA annotation tool 607 between gene and protein annotation tool 606B and domain annotation tool 608B, causing at least some of the data from the bioinformatics dataset to be first processed by tRNA annotation tool 607 prior to processing by domain annotation tool 608B. Once processing by tRNA annotation tool 607 completes, domain annotation tool 608B processes the bioinformatics dataset, and outputs its results to outputs 610B.

The processing of the bioinformatics dataset by the various bioinformatics processing tools of the initial and/or modified bioinformatics pipelines may result in a wide range of output types and amounts. For example, when the third bioinformatics processing tool replaces the second bioinformatics processing tool in the processing sequence, the final output may simply be the output of the third bioinformatics processing tool. In other cases, such as when the second bioinformatics processing tool and the third bioinformatics processing tool are operating in parallel, the outputs produced by the different tools may be provided separately or may be combined to generate a single, aggregated output. For additional examples of generated outputs, along with additional examples of many of the features discussed in this sub-section, see the Further Comments and/or Embodiments sub-section of this Detailed Description, below.

It should be noted that the bioinformatics pipelines discussed herein improve upon existing bioinformatics pipelines in many meaningful ways discussed throughout the various sub-sections of this Detailed Description. For example, by utilizing a self-morphing pipeline construction which leverages newer tools and/or tools better suited for the data analytics task at hand, various embodiments of the present invention produce more accurate pipeline results than those produced by existing pipelines. It should further be noted that many embodiments also improve the underlying technology used to execute the disclosed pipelines. For example, by allowing for bioinformatics pipelines to be modified in real time, embodiments of the present invention allow for bioinformatics pipeline workloads to be more seamlessly distributed across cloud computing environments, such as the environments discussed in The Hardware and Software Environments sub-section of this Detailed Description. Furthermore, the ability of embodiments of the present invention to process bioinformatics datasets through different bioinformatics processing tools, sometimes even in parallel, as part of a single bioinformatics pipeline reduces the amount of compute, memory, storage, and network resources needed to perform such processing over traditional configurations that would require multiple pipelines for processing.

### III. Further Comments and/or Embodiments

Various embodiments of the present invention recognize the following facts, potential problems and/or potential areas for improvement with respect to the current state of the art: (i) existing bioinformatics pipelines are typically deterministic in nature, and are not particularly suited to work on modern, agile, hybrid-cloud based resources; (ii) changes to bioinformatics pipelines typically require rewriting and relaunching—existing pipelines do not support real time decision making that can dynamically morph a pipeline depending on availability of resources, or on changes in objectives of processing.

Various embodiments of the present invention differ from existing pipeline management solutions in: (i) the definition of what a “pipeline” is, and (ii) the execution of such a pipeline using a machine learning based decision model such as an AI classifier. For example, existing “pipelines” tend to be defined by sets of pre-determined and finite steps that need to be performed in a certain order of precedence. These pipelines are specified at the user level, are fixed in their design, and are intended to achieve a fixed goal. And even when those pipelines are decoupled and executed on disparate, distributed computing resources, the underlying structure and order of the pipelines does not change. Embodiments of the present invention, however, define pipelines that are not fixed, but rather are dynamic/changing in nature. For example, various embodiments of the present invention allow for the dynamic inclusion of additional pipelines steps/tools that were not defined in an original pipeline. This self-morphic nature of a pipeline, executed using a machine learning/AI powered orchestration engine, is fundamentally different from the fixed pipelines of existing solutions.

For example, consider a user-defined pipeline that specifies that steps A, B, and C should be performed in a certain order: first A, then B, then C. Existing pipeline management solutions may execute A, B, and C in parallel or in a partially overlapping way, but would ultimately ensure that A, B, and C are performed without modifying the user-defined logic of precedence. Various embodiments of the present invention, however, are configured to modify a user-defined pipeline in ways not permitted by existing solutions, such as by summoning and executing additional steps D and E at run-time, even though they were not specified at the user level.

Various embodiments of the present invention provide a computer-based system for creating and managing self-morphing bioinformatics pipelines. Various embodiments provide for intelligent, real-time, autonomous control of pipeline size, depth, and shape, with considerations for available compute resources, performance, and/or desired runtime as well as tooling resource limitations or requirements. Various embodiments also provide for autonomous intelligent control of pipeline component methods/algorithms based on real-time monitoring and decision-making of intermediate outcomes/results, with considerations for desired ultimate output, quality control, filtering, and/or data integrity.

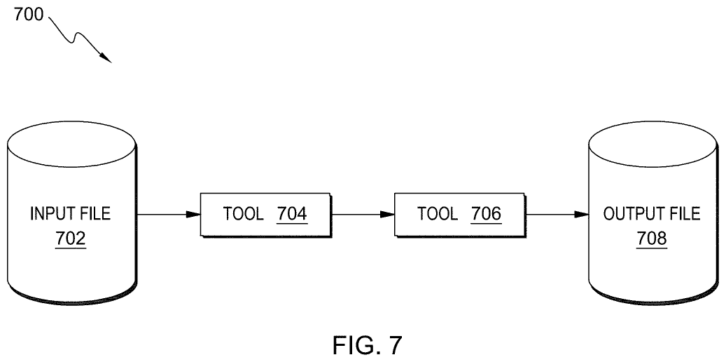

FIG. 7 is a block diagram showing a bioinformatics pipeline according to an embodiment of the present invention. As shown in FIG. 7, bioinformatics pipeline 700 includes input file 702, tool 704, tool 706, and output file 708. In this embodiment, input file 702 is processed by tool 704, generating an output. The output is then processed by tool 706, resulting in final output file 708. In this embodiment, the sending of input file 702 to tool 704, and the processing of input file 702 by tool 704, is also referred to as “Step 1” of pipeline 700, and the sending of the output of tool 704 to tool 706, the processing of the output of tool 704 by tool 706, and the resulting population of output file 708 with the output of tool 706, is also referred to as “Step 2” of pipeline 700. In some cases, Step 1 and Step 2 of pipeline 700 are somewhat rigid and pre-defined — however, in many cases, as will be discussed below, pipeline 700 is self-morphing, resulting in various on-the-fly changes during processing.

Referring to FIG. 7, in an example, a hospital has introduced transcriptome profiling for its patients using next-generation sequencing techniques. The computational effort to support this endeavor involves implementing an RNA-Seq pipeline that involves several tools running in a pre-defined fashion. In this particular example, pipeline 700 is a Tuxedo pipeline, input file 702 is an RNA-Seq dataset, tool 704 is the known Tophat tool, and tool 706 is the known Cufflinks tool. Step 1 in pipeline 700 requires read mapping using Tophat (tool 704). In this example, the read mapping is an independent event and is atomic in nature (that is, one input file is not dependent on another input file for the mapping operation to take place). Tophat (tool 704) takes one sequence at a time, maps the sequence, and produces corresponding output until all of the sequences are consumed. The output of Tophat (tool 704) is then consumed by Cufflinks (tool 706) for further downstream processing in Step 2.

Continuing the example, the hospital receives an instruction to extend their transcriptomic profiling to gather information about patients who may be affected by an emerging public health event (such as the COVID-19 pandemic). The hospital now needs to adjust its computational effort to handle possible cases relating to the public health event. In a traditional bioinformatics setup, the hospital would need to configure two bioinformatics pipelines—one for existing transcriptomics operations and the other for identifying cases relating to the public health event (for example, cases where the patient is COVID-19 positive). In both pipelines, Step 1—read mapping using Tophat (tool 704)—will be common, which means that the read mapping workload would be doubled (performed twice per case—one for each pipeline). Various embodiments of the present invention solve this problem by allowing pipeline 700 to morph in real time to cover activities that would typically be covered by the two separate pipelines in traditional bioinformatics configurations.

Still continuing the example, single pipeline 700 is employed instead of two separate pipelines. For all patients, Tophat (tool 704) is the first tool to be called. During processing, a reference database utilized by pipeline 700 may be appended with additional sequences that can be used to identify potential matches related to the public health event. If, at the read mapping stage (Step 1), matches related to the public health event are discovered, those matches are identified for specific bioinformatics processing in addition to the transcriptomic profiling discussed above. This identification is performed by an orchestration engine, which is configured to dynamically identify states in the pipeline, and, in the case of certain observations, launch additional “routes” in the pipeline that require calling extra tools that were not part of the original workflow. The orchestration engine thereby changes the shape of the workflow in a data driven manner, as opposed to the process driven manner of a traditional bioinformatics workflow. The orchestration engine can continue to morph the workflow as additional events are defined and identified.

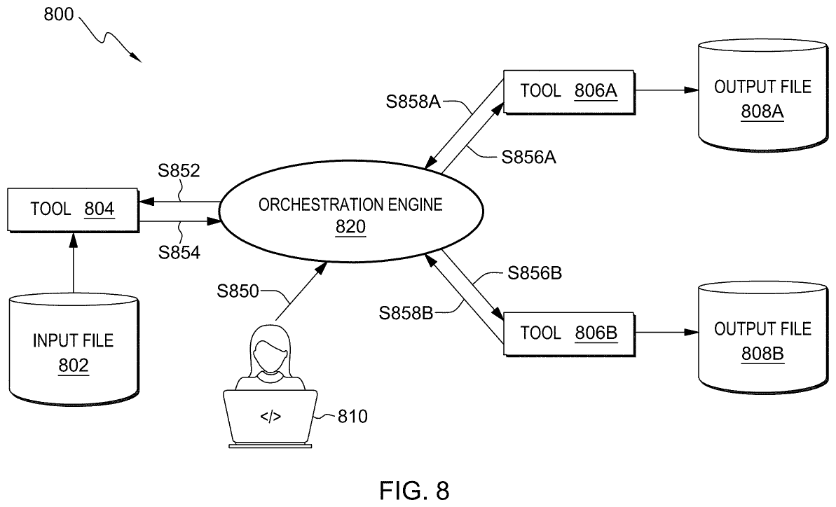

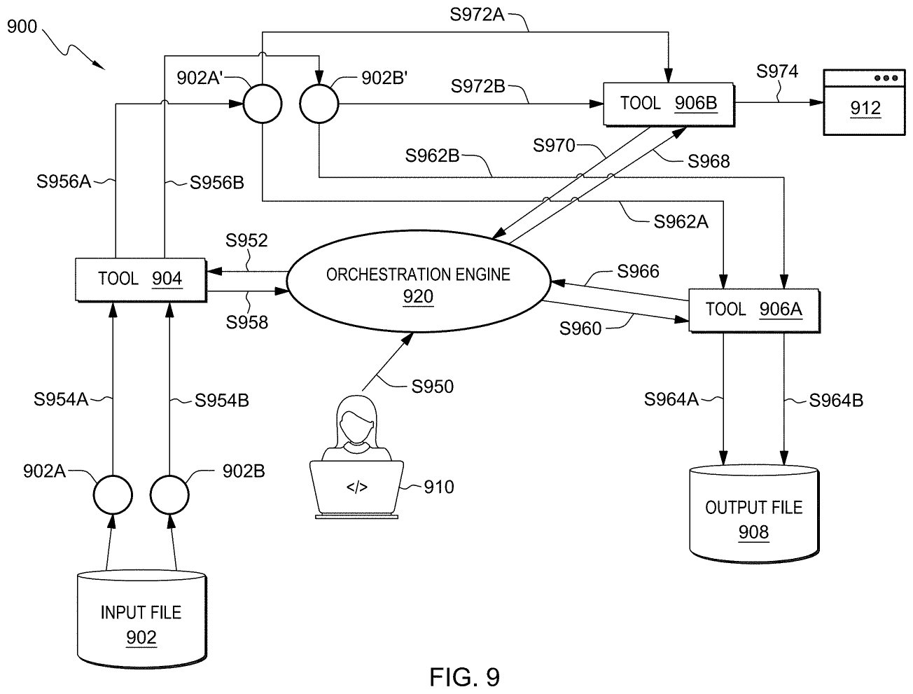

FIG. 8 is a block diagram showing a bioinformatics pipeline with an orchestration engine, according to an embodiment of the present invention. As shown in FIG. 8, bioinformatics pipeline 800 includes input file 802, tool 804, tool 806A, tool 806B, output file 808A, output file 808B, user 810, and orchestration engine 820. Additionally, FIG. 8 depicts method operations S850, S852, S854, S856A, S856B, S858A, and S858B, performed by various components of pipeline 800, such as orchestration engine 820, as will be discussed in further detail below.

In the embodiment depicted in FIG. 8, processing begins when user 810 submits (operation S850) a job to orchestration engine 820 for processing. The state of the job submitted by user 810 may be encapsulated in a variety of ways. For example, the job may be defined using a simplified grammar, such as an SQL-like grammar or a Domain Specific Language (DSL), which provides instructions on the “what” and the “how” of the job. For example, in a case where user 810 is requesting gene prediction and entity naming, the grammar may look as follows:

### Example Grammar

WITH GENE PREDICATION USE INPUT <file>USING CONSTRAINTS constraint1, constraints2
AFTER WITH ENTITY NAMING USING CONSTRAINTS constraint1, constraint2

In this example, constraints would be specific criteria to use for a given tool. The constraints would also be part of training a decision model for use by orchestration engine 820, as will be discussed below.

In other cases, instead of using a grammar such as the grammar described above, user 810 may define the job using a structured file, such as a YAML, JSON, or XML file, in a manner similar to the grammar. In still other cases, user 810 may define and/or encapsulate the state of the job using a user interface that exposes the information needed to execute a bioinformatics pipeline.

Referring still to the embodiment depicted in FIG. 8, once user 810 submits the job to orchestration engine 820 for processing, orchestration engine 820 uses a decision model to decide which tool to use to begin processing the job. In many cases, the model is a machine learning/artificial intelligence classifier, the training and use of which will be discussed in further detail below.

In the embodiment depicted in FIG. 8, orchestration engine 820 selects tool 804 to begin processing the received job. In operation S852, orchestration engine 820 allocates any required resources to tool 804, provides any required inputs, including input file 802, to tool 804, and sends an instruction to tool 804 instructing tool 804 to execute.

Tool 804 then processes input file 802 as instructed. Once complete, tool 804 notifies orchestration engine 820 that processing has completed. Orchestration engine 820 receives (operation S854) the notification from tool 804, and then determines the next tool for processing using the decision model based, for example, on the details of the job outlined in the provided file/grammar.

Using the example discussed above with respect to FIG. 7, orchestration engine 820 then determines that the output of tool 804 includes some sequences (for example, SARS-CoV-2 matches) that relate to the public health event. In this case, orchestration engine determines to send (operation S856A) the sequences relating to the public health event to tool 806A and to send (operation S856B) the remaining sequences to tool 806B. As with the selection of tool 804 discussed above, the selection of tool 806A and tool 806B may also include allocating any required resources to tool 806A and tool 806B, providing any required inputs to tool 806A and tool 806B, and sending instructions to tool 806A and tool 806B instructing tool 806A and tool 806B to execute.

Once tool 806A and tool 806B have completed processing, tool 806A and tool 806B notify orchestration engine 820 that processing has completed, and orchestration engine 820 receives the notifications from tool 806A (operation S858A) and tool 806B (operation S858B), respectively. Orchestration engine 820 then uses the decision model to determine whether to select a next tool for processing or to complete processing. In the present case, no additional processing is needed, and as such orchestration engine 820 directs tool 806A and tool 806B to output their results to output file 808A and output file 808B, respectively.

If orchestration engine 820 is unable to identify an already installed tool for processing, it may, in some cases, identify an alternative tool that requires additional installation. For example, orchestration engine 820 may identify a tool that requires installation, determine whether the system in which orchestration engine 820 operates meets requirements for installing the tool, and then prompt user 810 to approve installation of the tool if it meets the requirements. Otherwise, orchestration engine 820 may deliver an error message to user 810 indicating that no valid tools are available for processing the data.

As discussed above, orchestration engine 820 is configured to receive a job from user 810 and make various decisions about how to process the job—including when to send various datasets to various tools, and when to complete processing—in the overall context of bioinformatics pipeline 800. Orchestration engine 820 can be architected in a wide variety of ways and rely on a wide variety of information to make these decisions. An example of such an architecture is depicted in FIG. 9.

FIG. 9 is a block diagram showing another bioinformatics pipeline with an orchestration engine, according to an embodiment of the present invention. As shown in FIG. 9, bioinformatics pipeline 900 includes input file 902, input file records 902A and 902B, tool 904, tool 906A, tool 906B, output file 908, user 910, browser display 912, and orchestration engine 920. Additionally, FIG. 9 depicts method operations S950, S952, S954A, S954B, S956A, S956B, S958, S960, S962A, S962B, S964A, S964B, S966, S968, S970, S972A, S972B, and S974, performed by various components of pipeline 900, such as orchestration engine 920, as will be discussed in further detail below.

In the embodiment depicted in FIG. 9, processing begins when user 910 generates (operation S950) an instruction to start an experiment/job/pipeline and sends the instruction to orchestration engine 920 for processing. The instruction identifies input file 902 as the input file to be processed, where input file 902 includes, as an example, record 902A and record 902B.

Orchestration engine 920 identifies a first step of the pipeline involving tool 904, and starts the first step by sending (operation S952) an instruction to tool 904 to process record 902A and record 902B. Tool 904 then begins processing (operation S954A) record 902A. Tool 904 also being processing (operation S954B) record 902B, which can occur synchronously or asynchronously (that is, in parallel) with the processing (operation S954A) of record 902A. Tool 904 then outputs (operation S956A) record 902A′ for record 902A, outputs (operation S956B) record 902B′ for record 902B, and informs (operation S958) orchestration engine 920 that tool 904 has completed the processing of record 902A and record 902B.

Continuing with the embodiment depicted in FIG. 9, orchestration engine 920 identifies a second step of the pipeline involving tool 906A, and starts the second step by sending (operation S960) an instruction to tool 906A to process record 902A′ and record 902B′. Tool 906A then begins processing (operation S962A) record 902A′ and processing (operation S962B) record 902B′, which again, may occur synchronously or asynchronously. Tool 906A then outputs (operation S964A) information relating to the processing (operation S962A) of record 902A′ to output file 908, outputs (operation S964B) information relating to the processing (operation S962B) of record 902B′ to output file 908, and informs (operation S966) orchestration engine 920 that tool 906B has completed the processing of record 902A′ and record 902B′.

In addition to the above described behavior relating to pipeline 900, orchestration engine 920 also performs self-morphing capabilities, where additional tool 906B is dynamically called, and additional steps are introduced to pipeline 900, to consume the intermediate results of record 902A′ and record 902B′. In this embodiment, for example, tool 906B is a tool capable of displaying the intermediate results of record 902A′ and record 902B′ via browser display 912.

Continuing with the embodiment depicted in FIG. 9, tool 906B is configured to consume record 902A′ and record 902B′ and perform various visualization operations relating to record 902A′ and record 902B′ via browser display 912. While record 902A′ and record 902B′ are being processed by (or prior to processing by) tool 906A, orchestration engine 920 invokes tool 906B to check (operation S968) for the availability of tool 906B, and tool 906B, in turn, acknowledges (operation S970) that tool 906B is ready to process record 902A′ and record 902B′. Tool 906B processes (operation S972A) record 902A′ and processes (operation S972B) record 902B′ and displays (operation S974) the resulting visualization via browser display 912. It should be noted that in this embodiment, operations S968, S970, S972A, S972B, and S974 can be performed at any time with respect to the processing (operation S962A) of record 902A′ and the processing (operation S962B) of 902B′ by tool 906A, given the availability of record 902A′ and record 902B′.

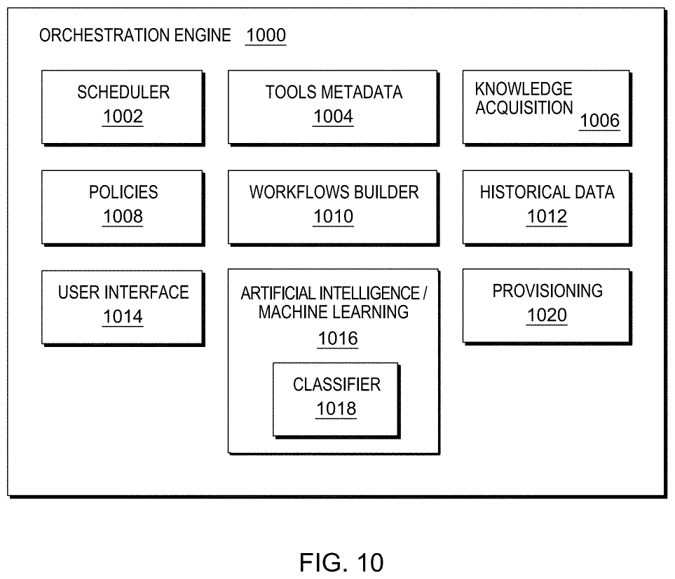

FIG. 10 is block diagram showing an orchestration engine architecture according to an embodiment of the present invention. As shown in FIG. 10, orchestration engine 1000 includes scheduler 1002, tools metadata 1004, knowledge acquisition 1006, policies 1008, workflows builder 1010, historical data 1012, user interface 1014, artificial intelligence/machine learning 1016 (which includes classifier 1018), and provisioning 1020. In the embodiment shown in FIG. 10: (i) scheduler 1002 includes information for launching workflows and monitoring workflows; (ii) tools metadata 1004 includes information about available tools, including release information, resource information, performance information, and repository information (that is, information regarding repositories in which tools are included); (iii) knowledge acquisition 1006 includes information relating to the scientific community, such as which tools are used and/or cited in various situations; (iv) policies 1008 includes resource requirements, quality information, and performance information for various tools; (v) workflows builder 1010 includes information for building workflows for processing in a bioinformatics pipeline; (vi) historical data 1012 includes information relating to previously executed workflows, including resources consumed and cost information; (vii) user interface 1014 includes interfaces (for example, graphical user interfaces, command line interfaces, and/or application programming interfaces) for collecting information from users and/or external programs/devices; (viii) artificial intelligence/machine learning 1016, which performs various types of machine learning, including active learning, and includes one or more decision models, including classifier 1018; and (ix) provisioning 1020 includes one or more modules responsible for provisioning and/or allocating compute and storage resources for a particular workflow.

Classifier 1018, a sub-component of artificial intelligence/machine learning 1016, plays an important part in intelligently and automatically determining which tools to use for a given set of data and constraints. A wide variety of different inputs/constraints can be used to train/generate classifier 1018, including much of the information discussed above in the preceding paragraph. Some examples of inputs/constraints that can be used to train/generate classifier 1018 include: (i) release information, where classifier 1018 could make decisions based on a newer release having better coverage or an older release mitigating a regression, as well as alerting users to stale or deprecated tools; (ii) resource information, where classifier 1018 could pick one tool over another based on whether the tool is supported (or better supported) by a current hardware and/or infrastructure configuration; (iii) performance information, where classifier 1018 could find tools that deliver faster responses, which could help with cost-based optimization (for example, some tools may require licenses or more resources and the more licenses and/or resources that are needed the more a user or institution will need to pay); (iv) repository information, which classifier 1018 could use to select tools from repositories that are actively developed and have a high number of followers and/or stars; (v) citation information, which classifier 1018 could use to select tools that are highly favored in the scientific domain; (vi) input type information, which classifier 1018 could use to select tools that meet the type of input the user is providing, with specific controls for file format requirements as well as data and domain relevance (orchestration engine 1000 could also provide a translation mechanism for inputs that may not quite match the user's input format but can be translated to a format which the tool supports); (vii) output type information, which classifier 1018 could use similarly to the input type information, but on output type requirements; (viii) configuration property information and command line parameter information, which classifier 1018 could use to determine the best set of supported features a tool provides based on what the user desires (this could be a catalog of tunable options the user could provide, and classifier 1018 could determine the tool best fitting those sets of options); (ix) known reference information, where some tools may require specific reference databases or flat files that contain information needed to perform certain lookups or calculations; and (x) domain knowledge information, where, in the event that orchestration engine 1000 is unable to deliver the tool or data necessary and user interaction is required, orchestration engine 1000 could employ active learning to gain domain knowledge of modifications to existing logic, data, and their relationships.

In yet another example, a FASTA dataset is provided to an orchestration engine for processing. In this embodiment, the dataset includes a full genome, and it includes a corresponding configuration file indicating that the genome should be annotated for genes, proteins, and domains. The orchestration engine first sends the dataset to a first annotator (or tool) that can identify genes and proteins within the genome. As a result, the first annotator produces a gene annotation which codes for proteins. The orchestration engine then examines the FASTA file and determines that another tool can be used to annotate RNA from the original genome. Here, the same input FASTA file would be used but a second tool—for example, one that can detect ncRNA—would be used. As a result of processing by the second tool, ncRNA would be identified, even if the ncRNA does not produce any proteins. The orchestration engine could then determine to examine the identified ncRNA for protein complementarity and/or protein binding, or for structural confirmations, such as hairpins. Then, for the previously annotated proteins, annotated by the first annotator, the orchestration engine could trigger domain annotation, which would involve examining the protein amino acids for smaller sub-components within the proteins that provide some biological activity. The domain annotation would use a database called PFAM to help locate specialized domains within the proteins discovered by the first annotator.

### IV. Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above—similar cautions apply to the term “embodiment.”

and/or: inclusive or; for example, A, B “and/or” C means that at least one of A or B or C is true and applicable.

Including/include/includes: unless otherwise explicitly noted, means “including but not necessarily limited to.”

User: includes, but is not necessarily limited to, the following: (i) a single individual human; (ii) an artificial intelligence entity with sufficient intelligence to act as a user; and/or (iii) a group of related users.

Data communication: any sort of data communication scheme now known or to be developed in the future, including wireless communication, wired communication and communication routes that have wireless and wired portions; data communication is not necessarily limited to: (i) direct data communication; (ii) indirect data communication; and/or (iii) data communication where the format, packetization status, medium, encryption status and/or protocol remains constant over the entire course of the data communication.

Receive/provide/send/input/output/report: unless otherwise explicitly specified, these words should not be taken to imply: (i) any particular degree of directness with respect to the relationship between their objects and subjects; and/or (ii) absence of intermediate components, actions and/or things interposed between their objects and subjects.

Automatically: without any human intervention.

Module/Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Computer: any device with significant data processing and/or machine readable instruction reading capabilities including, but not limited to: desktop computers, mainframe computers, laptop computers, field-programmable gate array (FPGA) based devices, smart phones, personal digital assistants (PDAs), body-mounted or inserted computers, embedded device style computers, application-specific integrated circuit (ASIC) based devices.

What is claimed is:

1. A computer-implemented method comprising:
identifying a bioinformatics dataset and instructions for processing the bioinformatics dataset, the instructions identifying a sequence of bioinformatics processing tools including at least a first bioinformatics processing tool followed by a second bioinformatics processing tool;
instructing the first bioinformatics processing tool to process the bioinformatics dataset in accordance with the instructions;
analyzing an output of the first bioinformatics processing tool, utilizing a machine learning based decision model, to determine a modification to the sequence of bioinformatics processing tools; and
instructing a third bioinformatics processing tool to process at least a first portion of the bioinformatics dataset in accordance with the determined modification.

2. The computer-implemented method of claim 1, wherein the determined modification replaces the second bioinformatics processing tool in the sequence of bioinformatics processing tools with the third bioinformatics processing tool for at least the first portion of the bioinformatics dataset.

3. The computer-implemented method of claim 2, further comprising:
instructing the second bioinformatics processing tool to process a second portion of the bioinformatics dataset in accordance with the sequence of bioinformatics processing tools.

4. The computer-implemented method of claim 1, wherein the determined modification adds the third bioinformatics processing tool to the sequence of bioinformatics processing tools after the first bioinformatics processing tool and before the second bioinformatics processing tool for at least the first portion of the bioinformatics dataset.

5. The computer-implemented method of claim 4, further comprising:
upon completion of the processing of the first portion of the bioinformatics dataset by the third bioinformatics processing tool, instructing the second bioinformatics processing tool to process the bioinformatics dataset.

6. The computer-implemented method of claim 1, wherein the determined modification adds a branch to the sequence of bioinformatics processing tools, the branch instructing parallel processing of at least the first portion of the bioinformatics dataset by both the second bioinformatics processing tool and the third bioinformatics processing tool.

7. The computer-implemented method of claim 6, further comprising:
instructing the second bioinformatics processing tool to process the bioinformatics dataset in parallel with the processing of the first portion of the bioinformatics dataset by the third bioinformatics processing tool.

8. The computer-implemented method of claim 1, wherein the machine learning based decision model includes an artificial intelligence classifier, and wherein the computer-implemented method further comprises:
training the artificial intelligence classifier utilizing historical bioinformatics datasets and a repository of bioinformatics processing tools, the repository of bioinformatics processing tools including at least one bioinformatics processing tool other than the bioinformatics processing tools included in the sequence of bioinformatics processing tools.

9. The computer-implemented method of claim 1, wherein:
the bioinformatics dataset is a genomic dataset; and
the first bioinformatic processing tool, the second bioinformatic processing tool, and the third bioinformatic processing tool are genomic annotation tools.

10. A computer program product comprising one or more computer readable storage media and program instructions collectively stored on the one or more computer readable storage media, the program instructions executable by one or more processors to cause the one or more processors to perform a method comprising:
identifying a bioinformatics dataset and instructions for processing the bioinformatics dataset, the instructions identifying a sequence of bioinformatics processing tools including at least a first bioinformatics processing tool followed by a second bioinformatics processing tool;
instructing the first bioinformatics processing tool to process the bioinformatics dataset in accordance with the instructions;
analyzing an output of the first bioinformatics processing tool, utilizing a machine learning based decision model, to determine a modification to the sequence of bioinformatics processing tools; and
instructing a third bioinformatics processing tool to process at least a first portion of the bioinformatics dataset in accordance with the determined modification.

11. The computer program product of claim 10, wherein the determined modification replaces the second bioinformatics processing tool in the sequence of bioinformatics processing tools with the third bioinformatics processing tool for at least the first portion of the bioinformatics dataset, and wherein the method further comprises:
instructing the second bioinformatics processing tool to process a second portion of the bioinformatics dataset in accordance with the sequence of bioinformatics processing tools.

12. The computer program product of claim 10, wherein the determined modification adds the third bioinformatics processing tool to the sequence of bioinformatics processing tools after the first bioinformatics processing tool and before the second bioinformatics processing tool for at least the first portion of the bioinformatics dataset, and wherein the method further comprises:
upon completion of the processing of the first portion of the bioinformatics dataset by the third bioinformatics processing tool, instructing the second bioinformatics processing tool to process the bioinformatics dataset.

13. The computer program product of claim 10, wherein the determined modification adds a branch to the sequence of bioinformatics processing tools, the branch instructing parallel processing of at least the first portion of the bioinformatics dataset by both the second bioinformatics processing tool and the third bioinformatics processing tool, and wherein the method further comprises:
instructing the second bioinformatics processing tool to process the bioinformatics dataset in parallel with the processing of the first portion of the bioinformatics dataset by the third bioinformatics processing tool.

14. The computer program product of claim 10, wherein the machine learning based decision model includes an artificial intelligence classifier, and wherein the method further comprises:
training the artificial intelligence classifier utilizing historical bioinformatics datasets and a repository of bioinformatics processing tools, the repository of bioinformatics processing tools including at least one bioinformatics processing tool other than the bioinformatics processing tools included in the sequence of bioinformatics processing tools.

15. The computer program product of claim 10, wherein:
the bioinformatics dataset is a genomic dataset; and
the first bioinformatic processing tool, the second bioinformatic processing tool, and the third bioinformatic processing tool are genomic annotation tools.

16. A computer system comprising:
one or more processors; and
one or more computer readable storage media;
wherein:
the one are more processors are structured, located, connected and/or programmed to execute program instructions collectively stored on the one or more computer readable storage media; and
the program instructions, when executed by the one or more processors, cause the one or more processors to perform a method comprising:
identifying a bioinformatics dataset and instructions for processing the bioinformatics dataset, the instructions identifying a sequence of bioinformatics processing tools including at least a first bioinformatics processing tool followed by a second bioinformatics processing tool;
instructing the first bioinformatics processing tool to process the bioinformatics dataset in accordance with the instructions;
analyzing an output of the first bioinformatics processing tool, utilizing a machine learning based decision model, to determine a modification to the sequence of bioinformatics processing tools; and
instructing a third bioinformatics processing tool to process at least a first portion of the bioinformatics dataset in accordance with the determined modification.

17. The computer system of claim 16, wherein the determined modification replaces the second bioinformatics processing tool in the sequence of bioinformatics processing tools with the third bioinformatics processing tool for at least the first portion of the bioinformatics dataset, and wherein the method further comprises:
instructing the second bioinformatics processing tool to process a second portion of the bioinformatics dataset in accordance with the sequence of bioinformatics processing tools.

18. The computer system of claim 16, wherein the determined modification adds the third bioinformatics processing tool to the sequence of bioinformatics processing tools after the first bioinformatics processing tool and before the second bioinformatics processing tool for at least the first portion of the bioinformatics dataset, and wherein the method further comprises:
upon completion of the processing of the first portion of the bioinformatics dataset by the third bioinformatics processing tool, instructing the second bioinformatics processing tool to process the bioinformatics dataset.

19. The computer system of claim 16, wherein the determined modification adds a branch to the sequence of bioinformatics processing tools, the branch instructing parallel processing of at least the first portion of the bioinformatics dataset by both the second bioinformatics processing tool and the third bioinformatics processing tool, and wherein the method further comprises:
instructing the second bioinformatics processing tool to process the bioinformatics dataset in parallel with the processing of the first portion of the bioinformatics dataset by the third bioinformatics processing tool.

20. The computer system of claim 16, wherein the machine learning based decision model includes an artificial intelligence classifier, and wherein the method further comprises:
training the artificial intelligence classifier utilizing historical bioinformatics datasets and a repository of bioinformatics processing tools, the repository of bioinformatics processing tools including at least one bioinformatics processing tool other than the bioinformatics processing tools included in the sequence of bioinformatics processing tools.
